(19) United States
(12) Patent Application Publication
(10) Pub. No.: US 2012/0053420 A1
(43) Pub. Date: Mar. 1, 2012

(54) ENDOSCOPIC LIGHT GUIDE AND ENDOSCOPE HAVING THE SAME

(75) Inventors: Tadashi KASAMATSU, Kanagawa-ken (JP); Koji Yoshida, Kanagawa-ken (JP)

(73) Assignee: FUJIFILM CORPORATION, Tokyo (JP)

(21) Appl. No.: 13/219,526

(22) Filed: Aug. 26, 2011

(30) Foreign Application Priority Data
Aug. 31, 2010 (JP) 194726/2010

(51) Int. Cl. A61B 1/06

(52) U.S. Cl. 600/182

(57) ABSTRACT

An endoscopic light guide for guiding illumination light to an observation target is equipped with: an optical fiber; and a radiation mode inducing means for causing propagation mode light propagating through the optical fiber to make side face radiation in the vicinity of an output facet of the optical fiber through which the propagation mode light exits, thereby converting the propagation mode light to radiation mode light such that the radiation mode light can be utilized as the illumination light.

### BACKGROUND OF THE INVENTION

1. Field of the Invention

The present invention relates an endoscopic light guide to be inserted into a body cavity to guide and illuminate illumination light on an observation target. The invention also relates to an endoscope having the same.

2. Description of the Related Art

Endoscope systems for observing tissues of body cavities have been widely known and an endoscope system that obtains a visual image by illuminating, for example, white light on an observation target in a body cavity and imaging the observation target, and displays the visual image on a monitor screen has been widely put into practical use.

Generally, the endoscope system described above uses an endoscopic light guide for guiding white light from a lamp light source to a body cavity as illumination light. Further, in order to realize more functional illumination (e.g., highlighting an affected area by the illumination of a specific wavelength) and for the sake of high intensity white illumination, suppression of heat generation, and smaller diameter, development of endoscopic light guides connected to laser sources as the light source for generating illumination light has been in progress as described, for example, in Japanese Unexamined Patent Publication No. 2005-328921.

The endoscopic light guide described in Japanese Unexamined Patent Publication No. 2005-328921 includes a fluorescent body at a distal end portion of the light guide and generates white light by exciting the fluorescent body with excitation laser light guided through the light guide. In this case, the size of the fluorescent body is 4 μm (about ten times of excitation wavelength) to 20 μm, and the light guide has a function to reduce unevenness of fluorescence by proactively causing forward scattering and evenly mixing the fluorescence spatially, in addition to the white light generation function. That is, the fluorescent body also acts as a diffuser plate based on the scattering.

Generally, laser light emitted from a laser light source may sometimes be harmful to human body due to high power density even if it is a small amount of emission. Accordingly, when laser light source is used as the illumination light source, it is preferable to lower the laser safety standard class as much as possible from the safety point of view for operation site.

In the mean time, reduction in the diameter of optical fibers used for endoscopic light guides has been advancing in view of handleability, durability, and downsizing. In this case, direct use of the optical fiber reduced in the diameter is dangerous from the view point of laser safety standard. Consequently, it is demanded that the emitting area and spread angle of output light be as large as possible.

As described above, in an endoscopic light guide that uses laser light as the illumination light, it is demanded that the spatial distribution of illumination be as uniform as possible, and the emitting area and spread angle of output light be as large as possible.

The method described in Japanese Unexamined Patent Publication No. 2005-328921, however, poses a problem that the extension range of emitting area is limited, although it may increase the emitting area to a certain extent. More specifically, based on the etendue conservation law that the product of spread angle of illumination light and emitting area at the output facet of the optical fiber is conserved, the spread angle of the illumination light and the emitting area are in a trade-off relationship so that the spread angle cannot be increased arbitrarily. In the mean time, the distance between the output facet of the optical fiber (primary light source) and the diffuser plate cannot be set arbitrarily due to the demand for reduced endoscopic light guide. Consequently, enlargement of emitting area of the secondary light source formed on the diffusion plate is inevitably limited. In such a case, only a small portion of the diffusion plate is used and, as a result, the size of illumination area is also limited.

The present invention has been developed in view of the circumstances described above, and it is an object of the present invention to provide an endoscopic light guide capable of lowering the laser safety standard class and an endoscope having the same.

### SUMMARY OF THE INVENTION

In order to achieve the objectives of the present invention, an endoscopic light guide of the present invention is an endoscopic light guide for guiding illumination light to an observation target, including:

an optical fiber; and

a radiation mode inducing means for causing propagation mode light propagating through the optical fiber to make side face radiation in the vicinity of an output facet of the optical fiber through which the propagation mode light exits, thereby converting the propagation mode light to radiation mode light such that the radiation mode light can be utilized as the illumination light.

In the endoscopic light guide of the present invention, the radiation mode inducing means may be a tapered portion formed at a predetermined portion of the optical fiber in the vicinity of the output facet and a core in the tapered portion has a tapering-off shape toward the output facet.

In this case, it is preferable that the tapered portion is configured to cause, if incident angle of the propagation mode light on the tapered portion is taken as θ₀ and critical angle of the optical fiber is taken as θ_{c}, propagation mode light having an incident angle θ₀ that satisfies formula (1) given below to make side face radiation, thereby converting the propagation mode light to radiation mode light.

θ₀/θ_{c}>0.2 (1)

The term “incident angle” of the propagation mode light on the tapered portion as used herein refers to an acute angle formed between travel direction of the propagation mode light and normal line of the input facet of the tapered portion which can be regarded as a propagation angle of the propagation mode light at the input facet. Here, the term “input facet of the tapered portion” refers to a cross-section of the optical fiber perpendicular to the optical axis at a point where the core diameter of the optical fiber starts to taper off (point where the core diameter starts to decrease).

Preferably, the tapered portion is configured to satisfy formula (2) given below, if length of the tapered portion in a direction of the optical axis is taken as L and propagated distance of the propagation mode light in a direction of the optical axis from a point at which the propagation mode light enters the tapered portion to a point at which the propagation mode light makes the side face radiation is taken as Lₚ.

Lₚ<L/2 (2)

The term “length of the tapered portion in a direction of the optical axis” as used herein refers to the distance from the input facet of the tapered portion to the output facet of the tapered portion (i.e., output facet of the optical fiber).

The term “propagated distance of the propagation mode light in a direction of the optical axis from a point at which the propagation mode light enters the tapered portion to a point at which the propagation mode light makes side face radiation” as used herein refers to the distance from the input facet of the tapered portion to a virtual cross-section of the optical fiber perpendicular to the optical axis at a point where the propagation mode light is converted to radiation mode light (point on the core-clad interface where the total reflection condition is no more met).

Preferably, the length of the tapered portion in a direction of the optical axis is within a range from 1 mm to 20 mm, and the taper angle of the tapered portion is within a range from 0.5 to 5 degrees.

The term “taper angle” as used herein refers to the angle formed between the generatrix of the side face of the tapered portion and the optical axis of the optical fiber.

In the mean time, the radiation mode inducing means may be a pressing member having at least one pressing terminal that presses a side face of the optical fiber in the vicinity of the output facet to generate microbending.

In this case, it is preferable that the pressing member has a plurality of the pressing terminals, and the pressing terminals are provided so as to press different positions shifted along a direction of the optical axis of the optical fiber as viewed from a direction perpendicular to the optical axis, and to press positions at the vertexes of a regular odd-gon inscribed in the optical fiber as viewed from the direction of the optical axis.

Further, it is preferable that radiation mode inducing means is equipped with a reflection member that guides the radiation mode light in a travel direction of the propagation mode light.

In this case, it is preferable that the reflection member has a reflection surface having a cylindrical shape or a frustoconical shape inversely tapered toward a distal end of the reflection surface and covering a periphery of the tapered portion, in which where the reflection surface has the fructoconical shape, the reflection surface covers the periphery of the tapered portion such that the distal end is disposed on the side of the output facet of the optical fiber.

Further, it is preferable that the radiation mode inducing means is equipped with is a coating member coating the side face of the tapered portion and being made of a material having a refractive index comparable to the refractive index of a material that constitutes the outermost portion of the tapered portion.

The term “comparable” to the refractive index of a material that constitutes the outermost portion of the tapered portion as used herein refers to that the refractive index of the coating member is equal to or close to the refractive index of the material that constitutes the outermost portion of the tapered portion so that reflection of light leaked out from the core is reduced at the interface between the coating member and the material that constitutes the outermost portion of the tapered portion.

An endoscope of the present invention is an endoscope, including:

the endoscopic light guide described above;

a light source connected to an input side of the endoscopic light guide and generates the illumination light; and

an imaging unit that receives light generated in the observation target as a result of illumination of the illumination light guided by the endoscopic light guide on the observation target and captures an image of the observation target.

The term “light generated in the observation target as a result of illumination of the illumination light” as used herein refers to, in the case, for example, where white light is used as the illumination light to obtain a visible light, reflection light of the white light, and in the case where excitation light is used as the illumination light to obtain a fluorescence image, fluorescence corresponding to the excitation light.

The endoscopic light guide for guiding illumination light to an observation target and the endoscope having the same according to the present invention includes, in particular, a radiation mode inducing means for causing the propagation mode light propagating through the optical fiber to make side face radiation in the vicinity of an output facet of the optical fiber through which the propagation mode light exits, thereby converting the propagation mode light to radiation mode light such that the radiation mode light can be utilized as the illumination light. This allows laser light to be extracted also from a portion of the optical fiber other than the output facet, thereby allowing a secondary light source having a large emitting area to be formed even with a thin optical fiber. This may lower the laser safety standard class for the endoscopic light guide and endoscope having the same even if a thin optical fiber is used.

### BRIEF DESCRIPTION OF THE DRAWINGS

FIG. 1 is an external view of an endoscope system having an endoscopic light guide according to a first embodiment of the present invention, illustrating a schematic configuration thereof.

FIG. 2 is a schematic view of a rigid insertion section, illustrating a composition thereof.

FIG. 3 is a schematic cross-sectional view of the rigid insertion section, illustrating an internal structure thereof.

FIG. 4A is a schematic cross-sectional view of a non-tapered optical fiber, illustrating an aspect in which a beam is outputted from the output facet of the fiber.

FIG. 4B is a schematic cross-sectional view of a typical tapered optical fiber, illustrating an aspect in which a beam is outputted from the output facet of the fiber.

FIG. 4C is a schematic cross-sectional view of a tapered optical fiber used for the light guide of the present invention, illustrating an aspect in which a beam is outputted from a portion adjacent to the output facet of the fiber.

FIG. 5 illustrates a schematic cross-sectional view of a tapered portion having a predetermined taper angle, illustrating an aspect in which propagation mode light is propagating in the tapered portion.

FIG. 6A schematically illustrates, in perspective view, a design example of reflection member.

FIG. 6B schematically illustrates, in perspective view, a design example of reflection member.

FIG. 6C schematically illustrates, in perspective view, a design example of reflection member.

FIG. 7A is a conceptual view illustrating an aspect in which radiation mode light is reflected by a reflection surface of a reflection member and focused in front.

FIG. 7B is a conceptual view illustrating an aspect in which radiation mode light is reflected by a reflection surface of a reflection member and focused in front.

FIG. 8A, is a schematic view of a radiation mode inducing means formed by inserting a tapered portion into the reflection member shown in FIG. 6A and filling a resin, illustrating a configuration thereof.

FIG. 8B is a schematic view of a radiation mode inducing means formed by inserting a tapered portion into the reflection member shown in FIG. 6C and filling a resin, illustrating a configuration thereof.

FIG. 9 is a schematic view of an imaging unit, illustrating a configuration thereof.

FIG. 10 is a schematic view of an image processing device and a light source device, illustrating a configuration thereof.

FIG. 11A is a schematic view of an optical fiber viewed from a direction perpendicular to the optical axis, illustrating that pressing members are mounted on the optical fiber.

FIG. 11B is a schematic view of an optical fiber viewed from a direction of the optical axis, illustrating that pressing members are mounted on the optical fiber.

### DESCRIPTION OF THE PREFERRED EMBODIMENTS

Hereinafter, embodiments of the present invention will be described with reference to the accompanying drawings, but it should be appreciated that the present invention is not limited to this. Note that each component in the drawings is not necessarily drawn to scale in order to facilitate visual recognition.

[First Embodiment of Endoscopic Light Guide and Endoscope Having the Same]

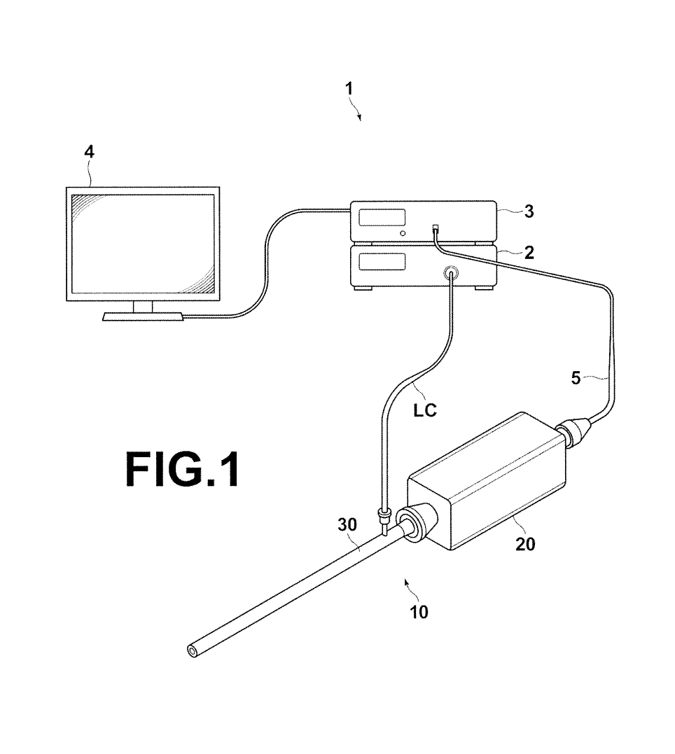

An endoscopic light guide or an endoscope having the same according to the first embodiment is used for an endoscope shown in FIG. 1. FIG. 1 is an external view of an endoscope system (rigid endoscope) which includes an endoscopic light guide (hereinafter, simply referred to as “light guide”) or an endoscope (rigid endoscope) having the endoscopic light guide according to the present embodiment.

### (Rigid Endoscope System)

As illustrated in FIG. 1, rigid endoscope system 1 of the present embodiment includes light source device 2 for emitting white illumination light and/or excitation light, rigid endoscope 10 for guiding and illuminating the illumination light and/or excitation light emitted from light source device 2 to an observation area and capturing a visible image based on reflection light reflected, by illumination of the white light, from the observation area and/or a fluorescence image based on fluorescence emitted, by illumination of the excitation light, from the observation area, image processing device 3 for performing predetermined processing on an image signal of the visible image and/or fluorescence image captured by rigid endoscope 10 and generating a visible image and/or a fluorescence image, and monitor 4 for displaying the visible image and fluorescence image of the observation area based on a display control signal generated in image processing device 3.

(Rigid Endoscope)

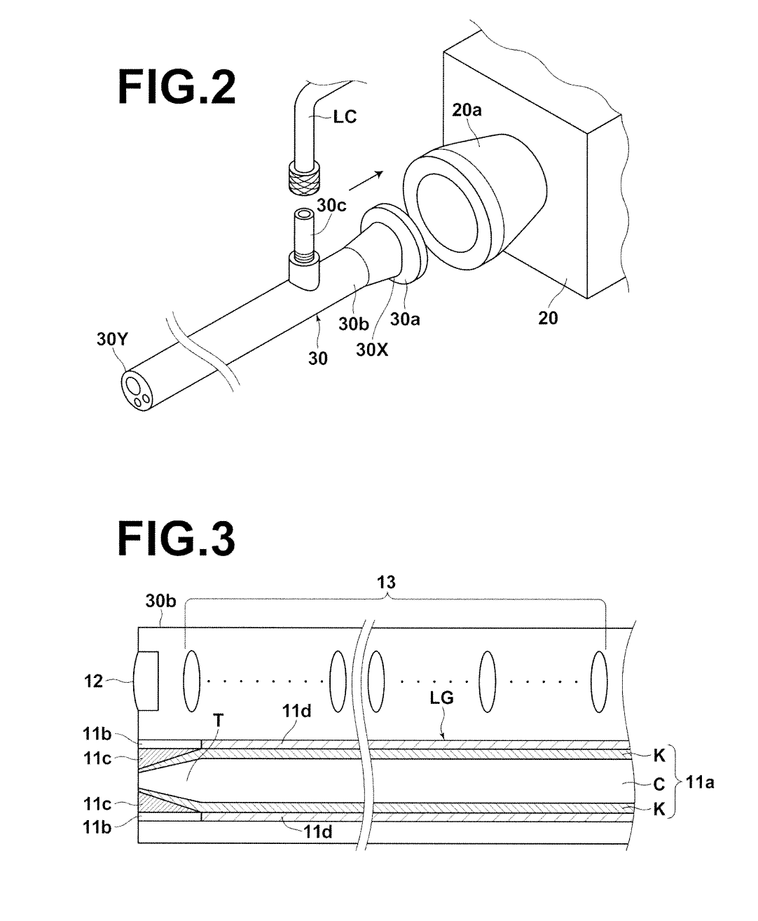

As illustrated in FIG. 1, rigid endoscope 10 includes rigid insertion section 30 to be inserted into an abdominal cavity and imaging unit 20 for capturing a visible image and fluorescence image of an observation target guided through the rigid insertion section 30. As shown in FIG. 2, rigid insertion section 30 and imaging unit 20 are detachably connected. Rigid insertion section 30 includes connection member 30a, insertion member 30b, and cable connection port 30c.

(Rigid Insertion Section)

Rigid insertion section 30 includes insertion member 30b for accommodating a light guide and an imaging optical system, connection member 30a used for connecting imaging unit 20, and cable connection port 30c for connecting an optical fiber LC for guiding light generated in light source 2.

Connection member 30a is provided at proximal end 30X of rigid insertion section 30 (insertion member 30b), and imaging unit 20 and rigid insertion section 30 are detachably connected by fitting connection member 30a into, for example, aperture 20a formed in imaging unit 20.

Cable connection port 30c is provided on the side face of insertion member 30b and an optical cable LC is mechanically connected to the port. This causes light source device 2 and rigid insertion section 30 to be optically linked through the optical cable LC.

Insertion member 30b is a member to be inserted into an abdominal cavity when imaging is performed in the abdominal cavity. Insertion member 30b is formed of a rigid material and has, for example, a cylindrical shape with a diameter of about 10 mm. FIG. 3 is a schematic cross-sectional view of the insertion member 30b, illustrating an internal structure thereof, i.e., an overall structure of the rigid insertion section 30. As illustrated in FIG. 3, insertion member 30b includes inside thereof a light guide LG having a multimode optical fiber for guiding and illuminating illumination light and/or excitation light emitted from light source 2 on an observation target, objective lens 12 for forming a visible image and a fluorescence image, lens group 13 for guiding the visible image and/or fluorescence image formed by objective lens 12. This causes the visible image and fluorescence image of the observation target inputted from the distal end 30Y (FIG. 2) are guided to imaging unit 20 on the proximal end side 30X through objective lens 12 and lens group 13.

(Light Guide)

A structure of the light guide LG provided inside of insertion member 30b will now be described in detail. As illustrated in FIG. 3, light guide LG includes optical fiber 11a, cylindrical reflection member 11b having a reflection surface surrounding the optical fiber in the vicinity of the output facet of the optical fiber, coating member 11c provided so as to fill between the output facet of optical fiber 11a and reflection member 11b, and a fixing member lid for fixing optical fiber 11a.

(Optical Fiber)

Optical fiber 11a is constituted by core C and clad K formed around the core. Illumination light and/or excitation light emitted from light source device 2 is inputted from one end of optical fiber 11a, guided through optical fiber 11a, and outputted from the other side, whereby guided to an observation target. There is not any specific restriction on the type and material of the optical fiber, and a multimode optical fiber is preferably used as the semiconductor laser, in general, is spatially oscillating in multimode when its output power is high and in order to obtain high coupling efficiency.

A predetermined portion in the vicinity of the output facet of optical fiber 11a is formed in a tapered shape so as to taper off toward the output facet. The predetermined portion of optical fiber 11a having the tapered shape is acts as a tapered portion T of an optical fiber and constituting a radiation mode inducing means of the present invention. The tapered portion T is formed in a tapered shape by heating a portion of optical fiber 11a and stretching the heated portion. That is, the tapered portion T of optical fiber 11a is formed such that the ratio between the core and clad diameters in the tapered portion T is identical to that in a non-tapered portion of optical fiber 11a. Preferably, in the light guide of the present invention, the length of the tapered portion T in a direction of the optical axis (tapered length) is 1 to 20 mm, and more preferably 2 to 5 mm. Here, the lower limit of “1 mm” is determined based on the fact that a minimum length of a tapered portion which can be produced is around 1 mm, although a short tapered portion, i.e., a tapered portion having a large taper angle may reduce a propagation distance that causes side face radiation. The upper limit of “20 mm” is determined based on the fact that, in the case of a long tapered portion, the light component that passes through the tapered portion by maintaining total reflection is increased and a maximum length of the tapered portion that can practically allow such component is about 20 mm. Preferably, the taper angle of the tapered portion is 0.5 to 5 degrees in consideration of limitations on the manufacturing, and more preferably 1 to 4 degrees. In the case where the tapered portion T is formed by the stretching process, the taper length and taper angle are substantially determined by the taper ratio of the tapered portion T in the stretching process. Therefore, the taper length and taper angle in the desired ranges may be obtained by performing the stretching process while appropriately setting the taper ratio. The term “taper ratio” as used herein refers to {(core diameter at input facet of the tapered portion)/(core diameter at output facet of the tapered portion)}×100%. Preferably, the taper ratio is less than 50%.

(Operation of Tapered Optical Fiber)

An operation of a typical tapered optical fiber will now be described by making comparison with that of a non-tapered optical fiber. Then, an operation of a tapered optical fiber used for the light guide LG of the present invention will be described by making comparison with those of the typical tapered optical fiber and non-tapered optical fiber.

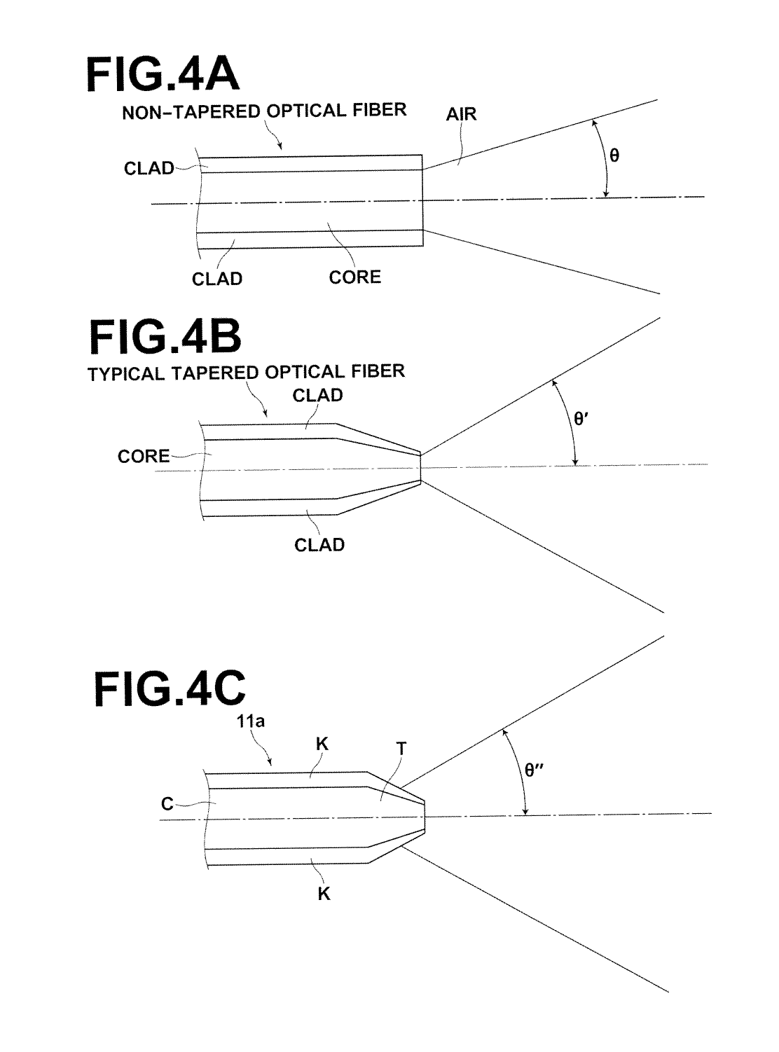

FIG. 4A schematically illustrates an operation of a non-tapered optical fiber, and FIG. 4B illustrates an operation of a typical tapered optical fiber, and FIG. 4C illustrates an operation of an optical fiber used for the light guide LG of the present invention.

Generally, a numerical aperture of the output facet of an optical fiber is represented by the formula (3) given below. The θ in the formula (3) given below is the θ shown in FIG. 4A which is a half spread angle of light outputted from the optical fiber. The n₁ and n₂ are refractive indices of the core and clad respectively.

NA=sin θ=\sqrt{n₁²−n₂²} =(3)

In the mean time, in optical fibers, in general, the product of the core diameter at the output facet and the half spread angle 8 of propagation mode light is constant. Therefore, the formula (4) given below holds true in any cross-section of the tapered portion in which the core diameter is continuously reduced toward the output facet for the tapered optical fiber as illustrated in FIG. 4B.

Beam Core Diameter(z)×θ(z)=Constant (4)

where, z is a variable indicating the position of an arbitrary cross-section of the tapered portion T.

Consequently, the half spread angle θ′ at the output facet of the tapered portion of the optical fiber shown in FIG. 4B is larger than the half spread angle θ at the output facet of the tapered portion of the optical fiber shown in FIG. 4A by an amount corresponding to the difference between the core diameters in FIGS. 4A and 4B.

Next, an operation of a tapered optical fiber used for the light guide LG of the present invention will be described. Tapered optical fiber 11a of the present invention is identical to that of FIG. 43 in that it has a tapered portion T in the vicinity of the output facet, but differs largely in the taper angle of the tapered portion T. In the case of FIG. 4B, the spread angle of propagation mode light outputted from the core of the output facet is merely increased using the relationship of formula (4) above. That is, in the case of FIG. 4B, it is assumed that the propagation mode light is radiated from the output facet of the tapered portion T (output facet of the optical fiber). In contrast, tapered optical fiber 11a of the present invention is structured such that propagation mode light entered into the tapered portion radiates from the side face of the tapered portion and is converted to radiation mode light by further increasing the taper angle. This allows laser light to be drawn from a portion of the optical fiber other than the output facet, as illustrated in FIG. 4C.

The reason why such side face radiation occurs is as follows. The propagation mode light entered into the tapered portion propagates in the core C of the tapered portion T by repeating total reflection at the core-clad interface of the tapered portion T. In the course of the propagation, the propagation angle of the light is increased by an amount of the taper angle each time the total reflection occurs and so as the incident angle on the interface. As a result, propagation mode light L1 incident on the interface in spite of having an incident angle greater than the critical angle becomes unable to make total reflection at the position of the interface and converted to radiation mode light L2. Note that the simple increase in the taper angle than that of a typical tapered optical fiber does not always cause the side face radiation described above. Whether or not the propagation mode light L1 makes side face radiation depends on, in particular, taper length, wavelength of light used, propagation angle of the propagation mode light L1, and the like, other than the taper angle. Therefore, in the case where the tapered portion T of the optical fiber is used as the radiation mode inducing means, as in the present embodiment, it is necessary to design the tapered portion T by taking into account the wavelength of light used, propagation angle of the propagation mode light L1, and the like.

In designing the tapered portion T, it is preferable that the tapered portion T is structured to cause, if incident angle of propagation mode light on the tapered portion is taken as θ₀, and the critical angle of the optical fiber is taken as θ_{c}, propagation mode light having an incident angle that satisfies the formula (1) above makes side face radiation, thereby converting the propagation light to radiation mode light. This may cause most of the energy of the light propagating through the optical fiber (approximately not less than 70% considering a typical incident angle θ₀ on the tapered portion) to make side face radiation.

(Side Face Radiation Judgment Method)

Specific judgment whether or not the propagation mode light L1 makes side face radiation is made by whether or not the formula (5) given below is satisfied, if the taper length is taken as L, and the propagated length in the direction of the optical axis from a point at which the propagation mode light L1 having a certain propagation angle θ₀ enters the tapered portion T to a point at which the propagation mode light L1 makes side face radiation is taken as Lₚ. In the case where the formula (5) is satisfied, the propagation mode light L1 makes side face radiation and the propagation mode light L1 is converted to radiation mode light L2.

Lₚ<L (5)

Hereinafter, the formula (5) will be described in detail.

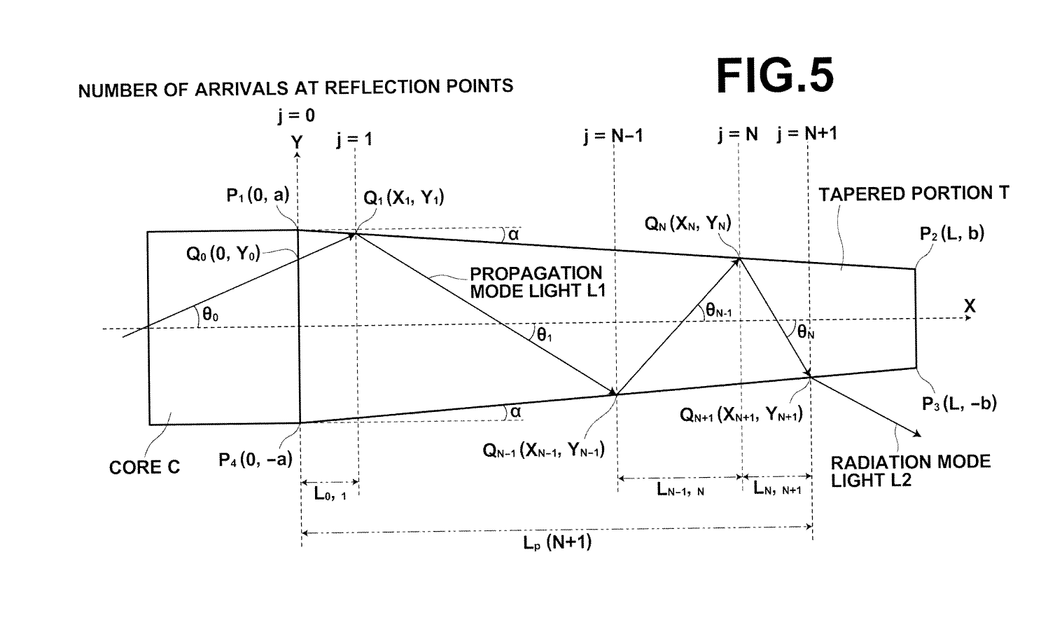

FIG. 5 is a schematic cross-sectional view of a tapered portion T having a taper angle α, illustrating an aspect in which propagation mode light L is propagating in the tapered portion. Note that FIG. 5 shows only a core without a clad for convenience sake. The core of the tapered portion is a portion defined by the coordinates P₁(0, a), P2(L, b), P3(L, −b), and P4(0, −a).

When propagation mode light L1 having a certain propagation angle θ₀ enters into the tapered portion T from a point Q₀ (0, Y₀) of the input facet of the tapered portion T, the propagation mode light L1 propagates toward the output facet of the tapered portion T by repeating total reflection at the side face of the core. The propagation angle θ₁ of the propagation mode light L1 after totally reflected once at Q₁ (X₁, Y₁) in FIG. 5 may be obtained by the formula (6) given below because the incident angle becomes equal to the propagation angle plus taper angle, and the propagation angle after reflection is equal to the reflection angle at the reflection surface plus taper angle.

|θ₁|=|θ₀|+2α (6)

The propagation angle θₙ after totally reflected n times may be obtain by the formula (7) given below because the side face of the core on which the reflection occurs is changed alternately.

θₙ=(−1)ⁿ·(|θ₀|+2αn) (7)

Therefore, the maximum possible total number of reflections N in which the propagation angle does not exceed the critical angle θ_{c} at the core side may be obtained by the formula (8) given below. Here, INT is an operator for rounding down decimal places of the calculation result in parentheses to obtain an integer.

N=INT((θ_{c}−θ₀)/2α) (8)

The critical angle θ_{c} may be defined as the formula (9) given below by the Snell's law in total reflection if the refractive index of the core of the tapered portion T is taken as n₁ and the refractive index of the clad is taken as n₂.

θ_{c}=cos⁻¹(n₂/n₁) (9)

Further, propagated distance of the propagation mode light L1 traveling toward the output facet of the tapered portion T in X direction from a side face of the core where the propagation mode light L1 is reflected to a next side face of the core where the propagation mode light L1 is reflected second time, i.e., the length of X component L_{j-1, j} of the distance between a point Qⱼ₋₁ where total reflection may possibly occur (reflection point Q) and the next reflection point Qj is represented by the formula (10) given below. Where, j represents the number of arrivals to reflection points Q and is an integer of 0 or greater. The maximum value of j is equal to the maximum possible total number of reflections N plus 1. j=0 represents the position of input facet of the tapered portion T which is taken as the origin of X axis (X0=0). The reflection point Qₙ₊₁ is not a point where total reflection occurs in a strict sense, but called as a reflection point for convenience sake.

L_{0,1}=X₁−X₀=X₁

L_{1,2}=X₂−X₁

- - -

L_{j-1,j}=Xⱼ−Xⱼ₋₁

- - -

L_{N-1,N}=X_{N}−X_{N-1}

L_{N,N+1}=X_{N+1}−X_{N} (10)

In this case, the length L_{P}(N+1) of X direction component of the propagated distance of the propagation mode light L1 through the core of the tapered portion to the reflection point Q_{N+1}, i.e., (N+1)^{th} reflection point is represented by the formula (11) given below. Where, L_{P}(j) represents the length of the X direction component of the propagated distance of the propagation mode light L1 through the core of the tapered portion T to j^{th} reflection point Qⱼ, and Zⱼ represents the width (length in Y direction) of the tapered portion T at j^{th} reflection point Qⱼ.

$$\begin{matrix}{L}_{p} ( N + 1 ) = {L}_{0 , 1} + {L}_{1 , 2} + {L}_{2 , 3} + … + {L}_{j - 1 , j} + … + {L}_{N , N + 1} \\ = \frac{{Z}_{0}}{\vert tan {θ}_{0} \vert + tan α} + \frac{{Z}_{1}}{\vert tan {θ}_{1} \vert + tan α} + \\ \frac{{Z}_{2}}{\vert tan {θ}_{2} \vert + tan α} + … + \frac{{Z}_{j - 1}}{\vert tan {θ}_{j - 1} \vert + tan α} + … + \\ \frac{{Z}_{N}}{\vert tan {θ}_{N} \vert + tan α} \\ = \frac{a - {Y}_{0}}{\vert tan {θ}_{0} \vert + tan α} + \frac{2 a - 2 {L}_{0 , 1} \cdot tan α}{\vert tan {θ}_{1} \vert + tan α} + \\ \frac{2 a - 2 ( {L}_{0 , 1} + {L}_{1 , 2} ) \cdot tan α}{\vert tan {θ}_{2} \vert + tan α} + … + \\ \frac{2 a - 2 \text{?} \text{?} \cdot tan α}{\vert tan {θ}_{j - 1} \vert + tan α} + … + \frac{2 a - 2 \text{?} \text{?} \cdot tan α}{\vert tan {θ}_{N} \vert + tan α}\end{matrix} \\ \text{?} \text{indicates text missing or illegible when filed} \qquad ( 11 )$$

The formula (11) may be derived by considering the relationship between the width Zⱼ₋₁ of the tapered portion T at (j−1)^{th} reflection point Qⱼ₋₁ and the width Zⱼ of the tapered portion T at j^{th} reflection point Q₁. That is, width Zⱼ₋₁ of the tapered portion T at (j−1)^{th} reflection point Qⱼ₋₁ may be represented by the formula (12) given below using the length L_{j-1,j} of X direction component of the distance between the reflection points Qⱼ₋₁ and Qⱼ.

Zⱼ₋₁=L_{j-1,j}·|tan θⱼ₋₁|+L_{j-1,j}·tan α (12)

In the mean time, Zⱼ₋₁ may be represented by the formula (13) given below using the length L_{P} of X direction component of the propagated distance of the propagation mode light L1 through the core of the tapered portion to the (j−1)^{th} reflection point Qⱼ₋₁.

$${Z}_{j - 1} = 2 a - 2 \text{?} \text{?} \cdot tan α \\ \text{?} \text{indicates text missing or illegible when filed} \qquad ( 13 )$$

From the formulae (12) and (13), the second to final terms of the formula (11) may be obtained as L_{j-1, j} is generally expressed as the formula (14) given below.

$${L}_{j - 1 , j} = \frac{2 a - 2 \text{?} \text{?} \cdot tan α}{\vert tan {θ}_{j - 1} \vert + tan α} \\ \text{?} \text{indicates text missing or illegible when filed} \qquad ( 14 )$$

The L_{P} in the formula (11) may be inductively obtained by obtaining an initial value Z₀. If the point of j=0 is taken as the input facet of the tapered portion T and the wide of the tapered portion T is taken as the core diameter at the input facet of the tapered portion T, Z₀ may be represented by the formula (15) given below. Consequently, the first term of the formula (11) may be obtained and hence L₂ may also be obtained.

Z₀=a−Y₀ (15)

As described above, in the case where the tapered portion T according to the present embodiment is used as the radiation mode inducing means, it is possible to design a tapered portion T properly such that the value of L_{P} obtained by the formula (11) satisfies the formula (5) while taking into account the actually used light source, optical fiber, and the like. This may cause propagation mode light L1 to make side face radiation at the tapered portion T of the optical fiber. Preferably, L_{P} is set to satisfy formula (2) above from the view point of forming an emitting area as large as possible. This allows radiation mode light L2 to be generated before propagation mode light L1 reaches the midpoint of the length L of the tapered portion T, thereby emitting area may be increased against the front side. So far, the operation of the tapered optical fiber 11a of the present invention has been described.

(Actual Calculation Example of Propagated Length in Optical Axis Direction Before Occurrence of Side Face Radiation)

Calculation examples fit for actual optical fibers are shown below. The optical fiber used is, by way of example, a multimode optical fiber with a fiber core diameter of 230 μm and a numerical aperture of 0.23. It is assumed that the optical fiber has been subjected to stretching process and has a tapered portion tapering off toward the output facet with a taper length of 5 mm and a core diameter of 40 μm at the output facet. In this case, the taper angle α is about 1.08 degrees. In the case where light is incident on the center (i.e., Y₀=0 in the formula (15) above) of the input facet of the tapered portion of the optical fiber described above with a propagation angle that satisfies the condition of θ₀/θ_{c}=0.4, the maximum number of possible total reflections is calculated as two. That is, the light does not satisfies the total reflection condition at the third reflection point (N+1=3) and becomes radiation mode. Here, the propagation length L_{P} (3) (sum of differences in distance L_{j-1, j,} j=1 to 3) is about 3.67 mm from the Table 1 shown below. That is, it is known that the light is radiated from the side face before propagating through the 5 mm tapered portion T.

| | | | | DIFFERENCE IN | PROPAGATED |
|---|---|---|---|---|---|
| | NUMBER | | | DISTANCE IN X | DISTANCE |
| NUMBER OF | OF | PROPA- | WAVE- | DIRECTION AT J^{TH} | TO J^{TH} |
| ARRIVALS AT | REFLEC- | GATION | GUIDE | REFLECTION | REFLECTION |
| REFLECTION | TIONS | ANGLE | WIDTH | POINTS | POINTS |
| POINTS j | n | θₙ | zⱼ μm | L_{j -} _{1, j} μm | Lₚ (j) μm |
| 0 | 0 | 0.06133 | 115.0000 | — | — |
| 1 | 1 | −0.09933 | 175.6488 | 1430.123 | 1430.123 |
| 2 | 2 | 0.13733 | 119.3935 | 1480.224 | 2910.348 |
| 3 | — | — | 90.5300 | 759.473 | 3669.821 |

The calculation example described above is just an example, and other calculation examples are also possible. Even in the case in which an optical fiber to be used and a propagation angle condition are changed, however, it is possible to properly design a tapered portion T such that a value L_{P} obtained by the formula (11) satisfies the formula (5) above through the procedure of the aforementioned calculation example.

(Reflection Member)

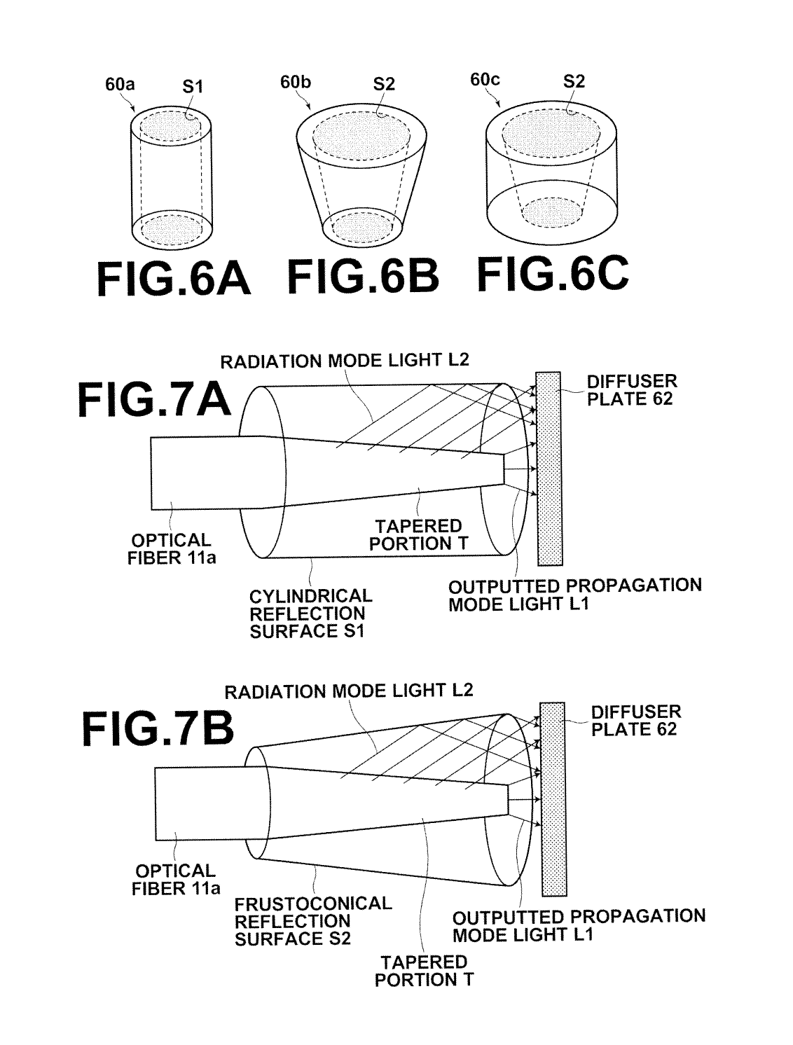

Reflection member 11b is a member having a reflection surface for focusing the radiation mode light L2 in a forward direction (travel direction of the propagation mode light L1). This allows the radiation mode light L2 to be efficiently used as illumination light. There is not any specific restriction on the shape of reflection member 11b and, for example, a shape 60a (FIG. 6A) which is cylindrical in both the outer side face and inner side face, a shape 60b (FIG. 6B) which is frustoconical inversely tapered toward a distal end of the reflection member 11b in both the outer side face and inner side face, a shape 60c (FIG. 6C) which is cylindrical in the outer side face and frustoconical inversely tapered toward a distal end of the reflection member 11c in the inner side face, and the like are preferably used. In view of ease of illuminance distribution adjustment of the illumination light, reflection surface S2 having a frustoconical shape inversely tapered toward the distal end of the reflection surface, as in FIG. 6B or 6C, is preferable. In such a case, it is preferable that the taper angle of the reflection surface (angle between the generatrix of the reflection surface and optical axis of optical fiber) is 2 to 3 degrees. There is not any specific restriction on the material of reflection member 11b and, for example, a metal such as gold or silver and glass may be used. In the case where a material having low reflectance properties, such as glass or the like, is used, a metal may be coated on the inner side face. The metal coating may be performed by deposition, plating, or the like. Further, as for the reflection member shown in FIG. 6A, for example, a so-called ferrule may be used.

There is not any specific restriction on the length of reflection member 11b along the optical axis of the optical fiber as long as it is around the taper length. There is not any specific restriction on the method of fixing optical fiber 11a to reflection member 11b and a method that provides a fixing member inside of reflection member 11b or a method that inserts optical fiber 11a into reflection member 11b and filling a resin or an adhesive. In the latter case, it is preferable that the resin or adhesive used acts also as a coating member, to be described later. Reflection member 11b shown in FIG. 3 is a cylindrical member 60a corresponding to that shown in FIG. 6A having a reflection surface on the inner side (not shown).

FIGS. 7A and 7B are conceptual view, illustrating an aspect in which radiation mode light L2 is reflected by a reflection surface of reflection member 11b and focused in front. In FIG. 7A or 7B, only the shape of the reflection surface of reflection member 11b is illustrated for convenience sake. FIG. 7A illustrates the case in which a cylindrical reflection surface 51 is placed. This corresponds to the case in which reflection member 60a of FIG. 6A is mounted on the tapered portion T of optical fiber 11a. FIG. 7B illustrates the case in which a frustoconical reflection surface S2 is placed. This corresponds to the case in which reflection member 60b of FIG. 6B or reflection member 60c of FIG. 6C is mounted on the tapered portion T of optical fiber 11a such that the distal end (wider end) of the reflection member 60b or 60c is disposed on the side of the output facet of the optical fiber 11a. As illustrated in FIGS. 7A and 7B, the reflection members are particularly useful for focusing radiation mode light L2 radiated from the side face of the optical fiber remote from the output facet at a position where illumination is required. In each of FIGS. 7A and 7B, diffusion plate 62 is provided in front of the output facet of optical fiber 11a. Such embodiment is preferable from the viewpoint of increasing the spread angle. The shape of the diffusion plate may be selected appropriately by considering a place where reflection member 11b is fixed.

(Coating Member)

Coating member 11c is provided around the tapered portion T of tapered optical fiber 11a and made of a material having a refractive index comparable to that of the clad. Preferably, the difference between the refractive index of coating member 11c and the refractive index of the clad is within ±0.5% and more preferably within ±0.4% in order to effectively cause side face radiation. These ranges are based on the fact that the refractive index difference is 0.2 to 0.3% for single mode and about 1% for multimode. Coating member 11c functions to prevent the radiation mode light L2 from forming clad mode. This may effectively cause radiation mode light L2 to make side face radiation to outside of the optical fiber. For example, a resin, such as UV curable resin, thermoset resin may be used for coating member 11c. Considering that the refractive index of the clad of a typical optical fiber is 1.45 to 1.46, a material having a refractive index of 1.44 to 1.47 is preferable as the material of coating member 11c. More specifically, for example, a UV curable adhesive (epoxy type) whose refractive index is adjustable from 1.45 to 1.50 is preferably used as the material of coating member 11c. In the case where the surface of coating member 11c is exposed to the air, surface roughness or flexure of not greater than micro level is formed on the surface of coating member 11c in order to prevent propagation mode from being formed inside of the coating member 11c. Coating member 11c does not necessarily cover the entire side face of the tapered portion T but, it is preferable that coating member 11c uniformly covers the side face of the tapered portion with respect to a circumferential direction centered on the optical axis from the viewpoint of equalizing illuminance distribution of radiation mode light L2 around the tapered portion.

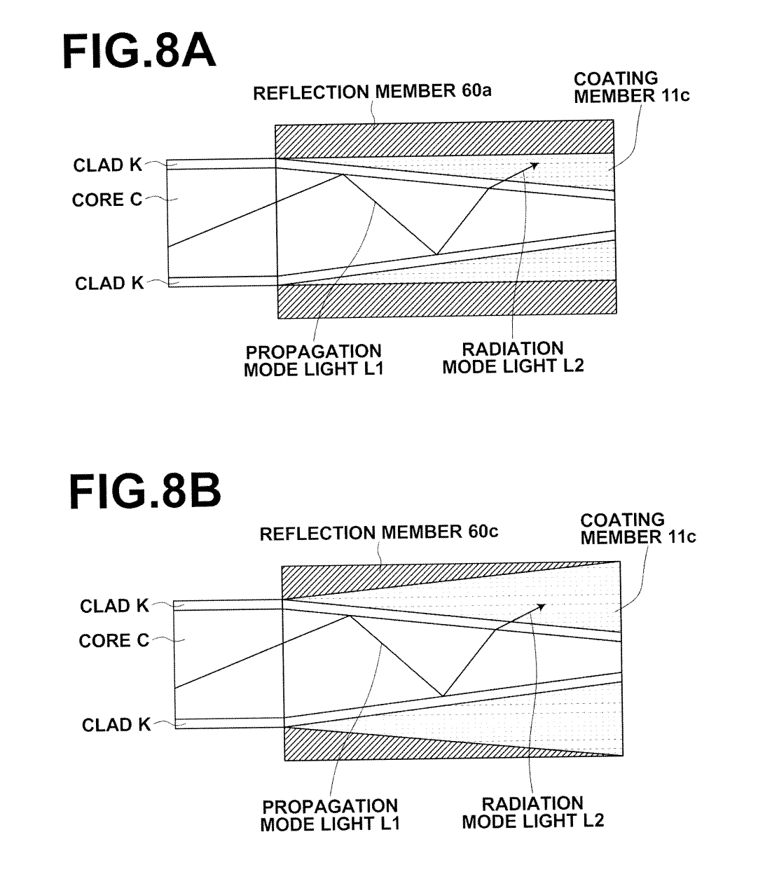

FIG. 8A is a schematic view of a radiation mode inducing means formed by inserting a tapered portion T into the reflection member 60a shown in FIG. 6A and filling a resin between the tapered portion T and reflection member 60a, illustrating a configuration thereof. FIG. 8B is a schematic view of a radiation mode inducing means formed by inserting a tapered portion T into the reflection member 60c shown in FIG. 6C and filling a resin between the tapered portion T and reflection member 60c, illustrating a configuration thereof. Such configurations are preferable because coating member 11c serves to fix the tapered portion T and reflection member.

### (Imaging Unit)

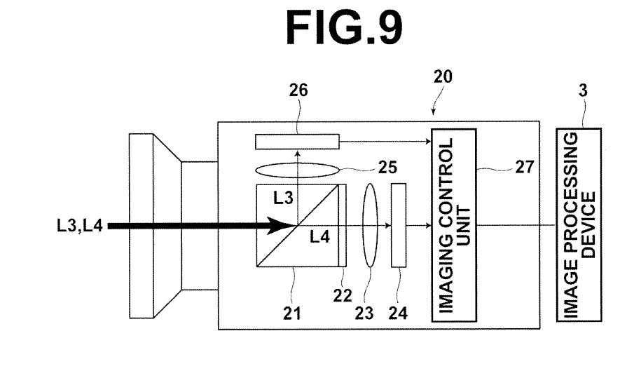

FIG. 9 is a schematic view of an imaging unit 20, illustrating a configuration thereof. Imaging unit 20 includes a first imaging system for imaging a fluorescence image of an observation target formed by lens group 13 in rigid insertion section 30 to generate a fluorescence image signal of the observation target, and a second imaging system for imaging a visible image of the observation target formed by lens group 13 in rigid insertion section 30 to generate a visible image signal of the observation target. These imaging systems are separated into two orthogonal optical axes by dichroic prism 21 having a spectral characteristic that reflects a visible image and transmits a fluorescence image.

The first imaging system includes excitation light cut filter 22 that cuts excitation light emitted from rigid insertion section 30 and transmitted through dichroic prism 21, first image forming optical system 23 that forms a fluorescence image L4 outputted from rigid insertion section 30 and transmitted through dichroic prism 21 and excitation light cut filter 22, and high sensitivity image sensor 24 that captures the fluorescence image L4 formed by first image forming optical system 23.

The second imaging system includes second image forming optical system 25 that forms a visible image L3 outputted from rigid insertion section 30 and reflected from dichroic prism 21, and image sensor 26 that captures the visible image L3 formed by second image forming optical system 25.

High sensitivity image sensor 24 detects light in a wavelength range of fluorescence image L4 with high sensitivity, converts the detected light to a fluorescence image signal, and outputs the fluorescence image signal. High sensitivity image sensor 24 is a monochrome image sensor.

Image sensor 26 detects light in the wavelength of visible image, converts the detected light to a visible image signal, and outputs the image signal. Color filters of three primary colors, red (R), green (G), and blue (B) or cyan (C), magenta (M), and yellow (Y) are arranged on the imaging surface of image sensor 26 in a Beyer or honeycomb pattern.

Imaging unit 20 further includes imaging control unit 27. Imaging control unit 27 performs CDS/AGC (correlated double sampling/automatic gain control) and A/D conversion on the fluorescence image signal outputted from high sensitivity image sensor 24 and the visible image signal outputted from image sensor 26, and outputs resultant image signals to image processing device 3 through cable 5 (FIG. 1)

(Image Processing Device)

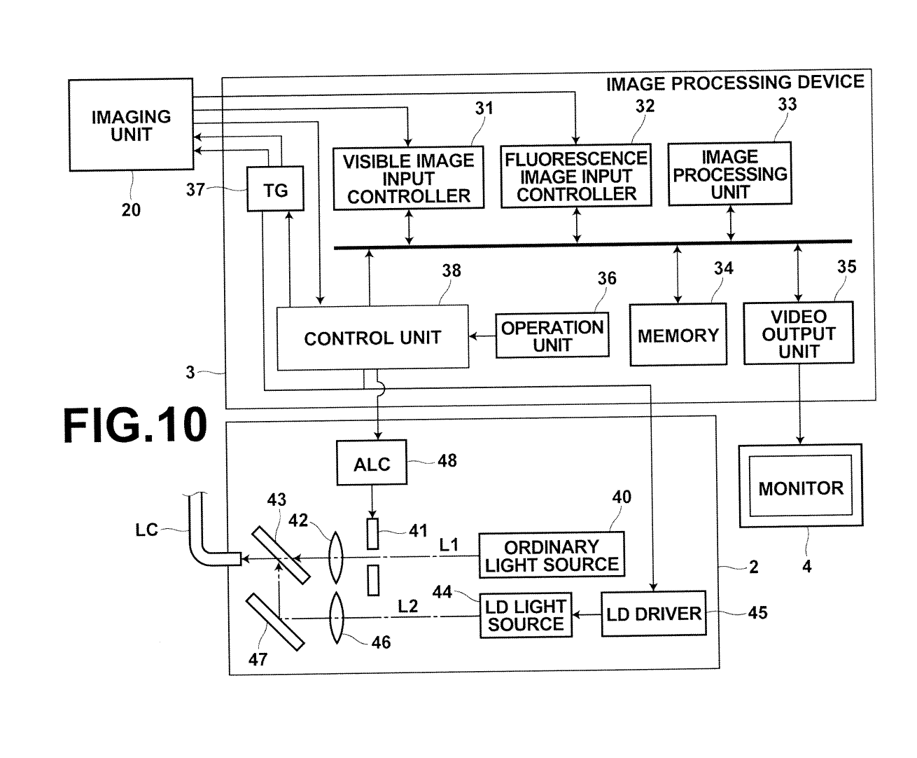

FIG. 10 is a schematic view of image processing device 3 and light source device 2, illustrating a configuration thereof.

As shown in FIG. 10, image processing device 3 includes visible image input controller 31, fluorescence image input controller 32, image processing unit 33, memory 34, video output unit 35, operation unit 36, TG (timing generator) 37, and control unit 38.

Visible image input controller 31 and fluorescence image input controller 32, each provided with a line buffer having a predetermined capacity, temporarily store visible image signals and fluorescence image signals with respect to each frame outputted from imaging control unit 27 of imaging unit 20 respectively. Then, the visible image signals stored in visible image input controller 31 and the fluorescence image signals stored in fluorescence image input controller 32 are stored in memory 34 via the bus.

Image processing unit 33 receives visible image signals and fluorescence image signals from memory 34 with respect to each frame, then performs predetermined processing on these image signals, and outputs the resultant image signals to the bus.

Video output unit 35 receives visible image signals and fluorescence image signals outputted from image processing unit 33 via the bus, then generates a display control signal by performing predetermine processing on the received signals, and outputs the display control signal to monitor 4.

Operation unit 36 receives input from the operator, such as various types of operational instructions and control parameters. TG 37 outputs drive pulse signals for driving high sensitivity image sensor 24 and image sensor 26 of imaging unit 20, and LD driver 45 of light source device 2, to be described later.

Control Unit 38 performs overall control of the system.

(Light Source Device)

light source device 2 includes visible light source 40 that emits visible light (white light) L1 constituted by a wide range of wavelengths from 400 to 700 nm, condenser lens 42 that condenses the visible light L1 emitted from visible light source 40, and dichroic mirror 43 that inputs the visible light L1 and excitation light L2, to be described later, to the input facet of the optical cable LC by transmitting the visible light L1 condensed by condenser lens 42 and reflecting the excitation light. As for visible light source 40, for example, a xenon lamp is used. Aperture 41 is provided between visible light source 40 and condenser lens 42, and the aperture amount is controlled based on a control signal from ALC (automatic light control).

Light source device 2 further includes LD light source 44 that emits 750 to 800 nm near infrared light as excitation light L2 for exciting an ICG (indocyanine green), which is a fluorescent pigment, to generate fluorescence, LD driver 45 that drives LD light source 44, condenser lens 46 that condenses the excitation light L2 emitted from LD light source 44, and mirror 47 that reflects the excitation light L2 condensed by condenser lens 46 to dichroic mirror 43.

In the present embodiment, light having the aforementioned wavelength range is used as the excitation light L2, but the wavelength is not limited to the range described above, and may be determined according to the type of fluorescent pigment administered to the subject or the type of living tissue for auto fluorescence.

As described above, the endoscopic light guide for guiding illumination light to an observation target and the endoscope having the same according to the present embodiment includes, in particular, a radiation mode inducing means for causing propagation mode light L1 propagating through the optical fiber to make side face radiation in the vicinity of an output facet of the optical fiber through which the propagation mode light exits, thereby converting the propagation mode light L1 to radiation mode light L2 such that the radiation mode light L2 can be utilized as the illumination light. This allows laser light to be extracted also from a portion of the optical fiber other than the output facet, thereby allowing a secondary light source having a large emitting area to be formed even with a thin optical fiber. This may lower the laser safety standard class for the endoscopic light guide and endoscope having the same even if a thin optical fiber is used.

(Design Change in Endoscopic Light Guide)

So far the description has been made of a case in which the endoscopic light guide of the present invention is applied to a rigid endoscope, but the application of the present invention is not limited to this and the invention is also applicable to a soft endoscope.

[Second Embodiment of Endoscopic Light Guide and Endoscope Having the Same]

An endoscopic light guide and an endoscope having the same according to a second embodiment will now be described. The endoscopic light guide and endoscope having the same according to the present embodiment have configurations substantially identical to those of the first embodiment. The second embodiment differs from the first embodiment in that the radiation mode inducing means is pressing member 64 for pressing the side face of an optical fiber in the vicinity of the output facet of the optical fiber to generate microbending. Therefore, components identical to those of the first embodiment will not be elaborated upon further here unless otherwise specifically required.

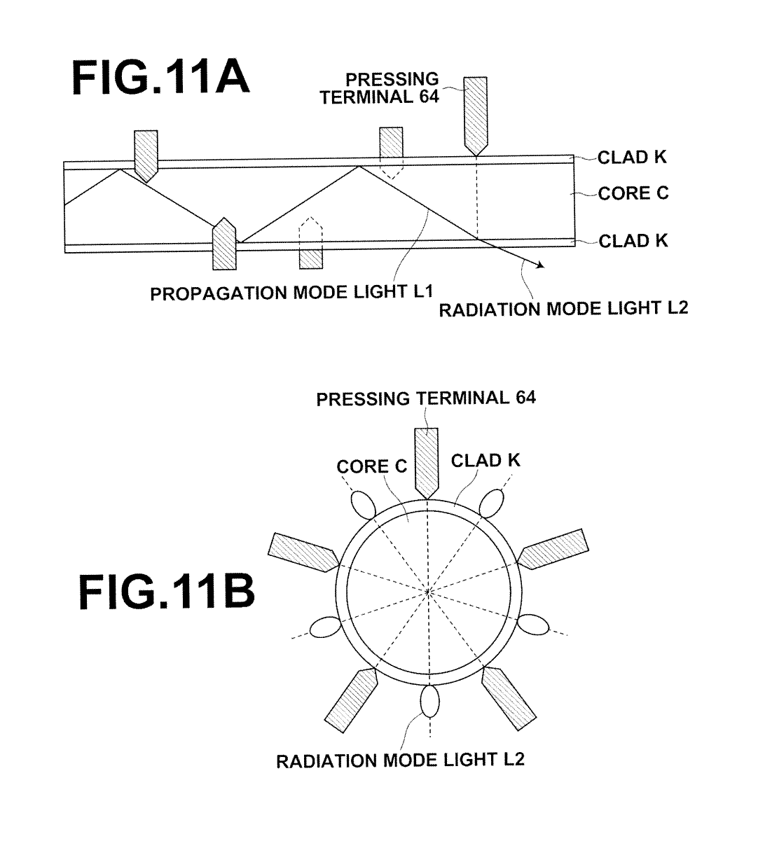

FIGS. 11A and 115 illustrate aspects in which a pressing member which is the radiation mode inducing means of the present embodiment is mounted on an optical fiber. FIG. 11A is a schematic view of the optical fiber as viewed from a direction perpendicular to the optical axis, illustrating that pressing members are mounted on the optical fiber. FIG. 11B is a schematic view of the optical fiber as viewed from a direction of the optical axis, illustrating that pressing members are mounted on the optical fiber.

As in the present embodiment, if an optical fiber is partially pressed, a microbending loss is generated and consequently radiation mode light L2 is generated. In this case, the pressing member 64 includes a plurality of pressing terminals 64, as illustrated in FIGS. 11A and 11B. Preferably, the plurality of pressing terminals 64 is provided so as to press different positions shifted along a direction of the optical axis as viewed from a direction perpendicular to the optical axis of the optical fiber and to press positions at vertexes of a regular odd-gon inscribed in the optical fiber as viewed from a direction of the optical axis. This does not cause a pressing terminal 64 to become a blockage against radiation mode light L2 generated due to other pressing terminals 64 and allows the radiation mode light L2 to be efficiently used as illumination light, as illustrated in FIGS. 11A and 11B. For example, in FIG. 11B, pressing terminals 64 are disposed so as to press the vertexes of regular pentagon inscribed in the optical fiber as viewed from a direction of the optical axis. A greater number of vertexes of regular odd-gon may result in more uniform illuminance distribution of the radiation mode light L2. The presser exerted by the pressing member 64 may be a small amount that deforms the core of the optical fiber by several micrometers. Further, the reflection member described above may be configured to include pressing terminals.

As described above, the endoscopic light guide for guiding illumination light to a observation target and the endoscope having the same according to the present embodiment includes, in particular, a radiation mode inducing means for causing propagation mode light L1 propagating through the optical fiber to make side face radiation in the vicinity of an output facet of the optical fiber through which the propagation mode light exits, thereby converting the propagation mode light L1 to radiation mode light L2 such that the radiation mode light L2 can be utilized as the illumination light, so that the present embodiment may provide advantageous effects identical to those of the first embodiment.

What is claimed is:

1. An endoscopic light guide for guiding illumination light to an observation target, comprising:
an optical fiber; and
a radiation mode inducing means for causing propagation mode light propagating through the optical fiber to make side face radiation in the vicinity of an output facet of the optical fiber through which the propagation mode light exits, thereby converting the propagation mode light to radiation mode light such that the radiation mode light can be utilized as the illumination light.

2. The endoscopic light guide of claim 1, wherein the radiation mode inducing means is a tapered portion formed at a predetermined portion of the optical fiber in the vicinity of the output facet and a core in the tapered portion has a tapering-off shape toward the output facet.

3. The endoscopic light guide of claim 2, wherein the tapered portion is configured to cause, if incident angle of the propagation mode light on the tapered portion is taken as θ₀ and critical angle of the optical fiber is taken as θ_{c}, propagation mode light having an incident angle θ₀ that satisfies formula (1) given below to make side face radiation, thereby converting the propagation mode light to radiation mode light.
θ₀/θ_{c}>0.2 (1)

4. The endoscopic light guide of claim 2, wherein the tapered portion is configured to satisfy formula (2) given below, if length of the tapered portion in a direction of the optical axis is taken as L and propagated distance of the propagation mode light in a direction of the optical axis from a point at which the propagation mode light enters the tapered portion to a point at which the propagation mode light makes the side face radiation is taken as Lₚ.
Lₚ<L/2 (2)

5. The endoscopic light guide of claim 3, wherein the tapered portion is configured to satisfy formula (2) given below, if length of the tapered portion in a direction of the optical axis is taken as L and propagated distance of the propagation mode light in a direction of the optical axis from a point at which the propagation mode light enters the tapered portion to a point at which the propagation mode light makes the side face radiation is taken as Lₚ.
Lₚ<L/2 (2)

6. The endoscopic light guide of claim 2, wherein:
the length of the tapered portion in a direction of the optical axis is within a range from 1 mm to 20 mm; and
the taper angle of the tapered portion is within a range from 0.5 to 5 degrees.

7. The endoscopic light guide of claim 3, wherein:
the length of the tapered portion in a direction of the optical axis is within a range from 1 mm to 20 mm; and
the taper angle of the tapered portion is within a range from 0.5 to 5 degrees.

8. The endoscopic light guide of claim 1, wherein the radiation mode inducing means is a pressing member having at least one pressing terminal that presses a side face of the optical fiber in the vicinity of the output facet to generate microbending.

9. The endoscopic light guide of claim 8, wherein:
the pressing member has a plurality of the pressing terminals; and
the pressing terminals are provided so as to press different positions shifted along a direction of the optical axis of the optical fiber as viewed from a direction perpendicular to the optical axis, and to press positions at the vertexes of a regular odd-gon inscribed in the optical fiber as viewed from the direction of the optical axis.

10. The endoscopic light guide of claim 1, wherein the radiation mode inducing means is equipped with a reflection member that guides the radiation mode light in a travel direction of the propagation mode light.

11. The endoscopic light guide of claim 10, wherein the reflection member has a reflection surface having a cylindrical shape or a frustoconical shape inversely tapered toward a distal end of the reflection surface and covering a periphery of the tapered portion, in which where the reflection surface has the frustoconical shape, the reflection surface covers the periphery of the tapered portion such that the distal end is disposed on the side of the output facet of the optical fiber.

12. The endoscopic light guide of claim 2, wherein the radiation mode inducing means is equipped with a coating member coating a side face of the tapered portion and being made of a material having a refractive index comparable to the refractive index of a material that constitutes the outermost portion of the tapered portion.

13. An endoscope, comprising:
the endoscopic light guide of claim 1;
a light source connected to an input side of the endoscopic light guide and generates the illumination light; and
an imaging unit that receives light generated in the observation target as a result of illumination of the illumination light guided by the endoscopic light guide on the observation target and captures an image of the observation target.
